(19) United States
(12) Patent Application Publication
(10) Pub. No.: US 2018/0289244 A1
(43) Pub. Date: Oct. 11, 2018

(54) ENDOSCOPE SYSTEM

(71) Applicant: HOYA CORPORATION, Tokyo (JP)

(72) Inventors: Yuya MASUKAWA, Tokyo (JP); Koji OHSE, Tokyo (JP)

(73) Assignee: HOYA CORPORATION, Tokyo (JP)

(21) Appl. No.: 15/767,007

(22) PCT Filed: Dec. 2, 2016
(86) PCT No.: PCT/JP2016/085869

§ 371 Date: Apr. 9, 2018

(30) Foreign Application Priority Data
Dec. 16, 2015 (JP) 2015-244836

(51) Int. Cl. A61B 1/00; A61B 1/018; A61B 1/05

(52) U.S. Cl. CPC A61B 1/00087; A61B 1/00011; A61B 1/01; A61B 1/05; A61B 1/00135; A61B 1/018

(57) ABSTRACT

An endoscope system is constituted by a processor and an endoscope. The processor includes: a ground terminal that is provided on an outer cover surface of a housing; a feedback terminal that is provided on the outer cover surface, and is connected to the ground terminal via an external capacitor arranged outside the housing; and a relay wiring line that is connected to the feedback terminal, and is arranged in the housing. The endoscope includes: a connector portion that is detachably attached to the processor; a channel into which a treatment tool is inserted; and a feedback wiring line that connects a metal member located in the vicinity of the channel to the connector portion. In this configuration, when the connector portion is attached to the processor, the feedback wiring line and the relay wiring line are connected to each other.

### TECHNICAL FIELD

The present invention relates to an endoscope system.

### BACKGROUND ART

Endoscope systems are known in which an endoscope is inserted into a human lumen such as the esophagus or the intestine, and an operator performs treatment while observing the inside of the lumen. With this type of endoscope system, the operator treats an affected site in the lumen by inserting a treatment tool such as a pair of forceps or a surgical knife into the endoscope so that the treatment tool protrudes from the distal end of the endoscope. Electrosurgical devices for cutting out an affected site and stopping bleeding are known as the treatment tool that is used.

For example, JP 2007-202654A (hereinafter, referred to as “Patent Document 1”) discloses, as such an endoscope system, an endoscope system that uses an electrosurgical knife. If an electrosurgical device such as an electrosurgical knife is used, electromagnetic waves generated in the electrosurgical device may cause an induced electric current to flow through a metal member of an endoscope. The induced electric current appears as noise on a captured image of a subject. Accordingly, in the endoscope system disclosed in Patent Document 1, the endoscope is provided with a feedback terminal for allowing the induced electric current to escape. By connecting this feedback terminal to, for example, a ground of a processor, an electric current flowing through the metal member is allowed to escape to the ground. Note that, because the electric current flowing through the metal member contains a high-frequency component, a capacitor that allows the high-frequency current to escape to the ground is usually arranged between the feedback terminal and the ground, instead of the feedback terminal being directly connected to the ground.

### SUMMARY OF INVENTION

In the electronic endoscope system disclosed in Patent Document 1, the feedback terminal is provided on an outer cover surface of the endoscope. Accordingly, when the used endoscope is washed or the endoscope is carried, an insertion portion of the endoscope that is to be inserted into a lumen may come into contact with the feedback terminal. The insertion portion is covered by a protective tube in order to protect wiring lines and the like that are provided inside of the insertion portion. This protective tube is made of a soft material such as a thin resin so that the insertion portion can easily bend. Therefore, there is the risk that the protective tube will be damaged if the insertion portion comes into contact with the feedback terminal.

The present invention was made in view of the above-described circumstances, and it is an object thereof to provide endoscope system that is appropriate for suppressing damage to the endoscope.

According to one embodiment of the present invention, an endoscope system is constituted by a processor and an endoscope, the processor including: a ground terminal that is provided on an outer cover surface of a housing; a feedback terminal that is provided on the outer cover surface, and is connected to the ground terminal via an external capacitor that is arranged outside the housing; and a relay wiring line that is connected to the feedback terminal, and is arranged in the housing, and the endoscope including: a connector portion that is detachably attached to the processor; a channel into which a treatment tool is inserted; and a feedback wiring line that connects a metal member that is located in the vicinity of the channel to the connector portion. In this configuration, when the connector portion is attached to the processor, the feedback wiring line and the relay wiring line are connected to each other, and thus the metal member is connected to the ground terminal via the feedback wiring line, the relay wiring line, the feedback terminal, and the external capacitor.

According to the embodiment of the present invention, for example, when the endoscope is connected to the processor via the connector portion, the metal member of the endoscope is grounded via the feedback wiring line, the relay wiring line, and the capacitor. Accordingly, there is no need to provide the endoscope with a terminal to which a wiring line for grounding the metal member is connected, and that is separate from the connector portion. It is thus possible to suppress damage to the endoscope that may be caused by the endoscope coming into contact with the terminal.

Furthermore, according to the embodiment of the present invention, the endoscope may have a rod-shaped insertion portion that is flexible and is to be inserted into a lumen of a patient, for example. In this case, the metal member may be arranged in the insertion portion, and may have a shape of an elongated tube that extends in a longitudinal direction of the insertion portion.

Furthermore, according to the embodiment of the present invention, the external capacitor may be detachably connected to the ground terminal and the feedback terminal, for example.

According to such a configuration, it is easy to replace the capacitor being used with one that corresponds to an electrosurgical device or a high-frequency power supply that is to be used.

According to the embodiment of the present invention, an endoscope system that is appropriate for suppressing damage to the endoscope is provided.

### BRIEF DESCRIPTION OF DRAWINGS

FIG. 1 is a diagram illustrating a configuration of an endoscope system according to an embodiment of the present invention.

### DESCRIPTION OF EMBODIMENTS

Hereinafter, an endoscope system according to an embodiment of the present invention will be described with reference to the drawing.

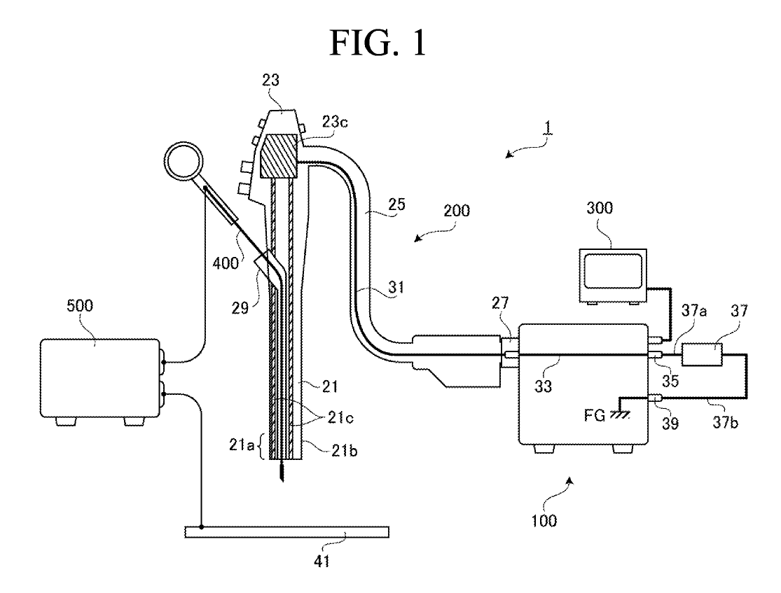

FIG. 1 is a diagram illustrating a configuration of an endoscope system 1 according to the present embodiment. The endoscope system 1 includes a processor 100, endoscope 200, a monitor 300, an electrosurgical device 400, and a high-frequency power supply 500.

The endoscope 200 has an insertion portion 21 that is to be inserted into a lumen of a patient, a control body 23 that is operated by an operator, a universal tube 25 that extends from the control body 23, and a connector portion 27 that is detachably connected to the processor 100. Furthermore, in the endoscope 200, a light guide is arranged extending from the connector portion 27 to a distal end 21a of the insertion portion 21, and guides irradiation light with which a subject is irradiated. In FIG. 1, the light guide is not shown.

The processor 100 has various types of electronic circuits for controlling the operation of the endoscope system 1, a light source unit for emitting illumination light for illuminating a subject, and an operation panel for accepting an operation made by the operator. Note that in FIG. 1, the electronic circuits, the light source unit, and the operation panel are not shown. The operation panel is used to change parameters relating to the operation of the endoscope system 1. The illumination light emitted from the light source unit is incident on the endoscope 200 via the connector portion 27. The illumination light incident on the endoscope 200 from the processor 100 is guided in the light guide, and exits from the distal end 21a of the insertion portion 21, and the subject is irradiated with the illumination light.

The insertion portion 21 of the endoscope 200 has an elongated rod shape, and the distal end 21a thereof is bent in accordance with an operation made on the control body 23. The part of the insertion portion 21 other than the distal end 21a is flexible. Furthermore, a protective tube 21b covers the surface of the insertion portion 21 in order to protect wiring lines and structures that are provided inside thereof, and to prevent liquid or contaminants from entering. The protective tube 21b is made of a soft material such as a thin resin so that the flexibility of the insertion portion 21 is not impaired. The operator inserts the insertion portion 21 into the lumen of the patient while bending the insertion portion 21. The distal end 21a of the insertion portion 21 is provided with an image sensor (not shown) for capturing an image of a subject in the lumen. Image signals of the subject captured by the image sensor are input to the processor 100 via the connector portion 27.

The processor 100 performs predetermined signal processing on the image signals of the subject that were input from the endoscope 200, and generates video signals. The video signals are output to the monitor 300, and thereby the monitor 300 displays the captured image of the subject.

The endoscope 200 has a treatment tool channel 29 through which various types of treatment tools are inserted. The treatment tool channel 29 is arranged in the insertion portion 21, and one end of the treatment tool channel 29 is placed in the vicinity of the control body 23, and the other end thereof is placed at the distal end 21a of the insertion portion 21. The operator inserts a treatment tool into the treatment tool channel 29 from the control body 23 side so that the treatment tool protrudes from the distal end 21a of the insertion portion 21. Accordingly, the operator can operate the treatment tool while observing a captured image displayed on the monitor 300, and treat an affected site in the lumen.

For example, the electrosurgical device 400 is employed as the treatment tool that is inserted through the treatment tool channel 29. The, electrosurgical device 400 is connected to the high-frequency power supply 500 that supplies a high-frequency current. The electrosurgical device 400 is activated by the supply of the high-frequency current, and is used in treatments such as those where all incision is made in an affected site, and clotting treatments.

The types of the electrosurgical device 400 are broadly classified into a monopolar type such as an electrosurgical knife, and a bipolar type such as electrosurgical forceps. The electrosurgical device 400 shown in FIG. 1 is a monopolar type electrosurgical device 400. When the monopolar type electrosurgical device 400 is used, a patient return electrode 41 is attached to the epidermis of the patient. The patient return electrode 41 is connected to the high-frequency power supply 500. When the electrosurgical device 400 comes into contact with a body tissue in the lumen of the patient, an electric current supplied to the electrosurgical device 400 flows to the patient return electrode 41 via the body of the patient. As a result of the high-frequency current flowing in this way, joule heat is generated, and can be used to treat the affected site.

The following will describe a case where, for example, electrosurgical forceps are used as a bipolar type electrosurgical device 400. The electrosurgical forceps include two holding portions that pinch an affected site. The two holding portions are independently connected to the high-frequency power supply 500. If an affected site is pinched by the two holding portions in a state in which the electrosurgical forceps are supplied with a high-frequency current, the high-frequency current will flow from one of the two holding portions to the other one via the affected site. Accordingly; the affected site that is pinched by the holding portions can be treated. Note that, when the bipolar type electrosurgical device 400 is used, no patient return electrode 41 is used.

Both cases where the monopolar type electrosurgical device 400 used and where the bipolar type electrosurgical device 400 is used share the fact that the electrosurgical device 400 generates Joule heat due to a high-frequency current. When the electrosurgical device 400 generates Joule heat, electromagnetic noise may occur from the electrosurgical device 400. This electromagnetic noise has the potential to generate an induced electric current in some of components that are located in the vicinity of the treatment tool channel 29, including the image sensor, a wiring line and an electronic circuit that are connected to the image sensor, and a metal member 21c that is used in the insertion portion 21 of the endoscope 200. If an induced electric current occurs in the image sensor, or a wiring line or an electronic circuit that is connected to the image sensor, there is the risk that noise will occur in a captured image of a subject. Furthermore, if an induced electric current is generated in the metal member 21c that is used in the insertion portion 21 of the endoscope 200, there is the risk that the operator will receive an electric shock when coming into contact with the metal member 21c. Accordingly, the insertion portion 21 is covered by the protective tube 21b that does not allow electricity to pass therethrough, and is configured to prevent an operator from coming into contact with the metal member 21c. Furthermore, in order to more reliably prevent ate operator from receiving an electric shock, the metal member 21c is grounded via the processor 100. Specifically, the metal member 21c is connected to a frame ground FG of the processor 100. The frame ground FG is, for example, a metal member in a housing, a chassis, or the like of the processor 100.

The following will describe a configuration in which the metal member 21c of the insertion portion 21 is grounded according to the present embodiment. The metal member 21c has the shape of an elongated tube that extends in a longitudinal direction of the insertion portion 21. The metal member 21c is arranged so as to cover the light guide and the treatment tool channel 29, thus protecting them. Furthermore, this metal member 21c employs, for example, a spiral tube that is made of metal. The spiral tube that is made of metal is obtained by forming a thin metal plate into a coil shape, and is flexible. Accordingly, even if the metal member 21c is arranged in the insertion portion 21, the insertion portion 21 can be flexible. The metal member 21c is connected to a plate-shaped metal base material 23c that is provided in the control body 23. The metal base material 23c is used to support an angulation operation knob (not shown) of the control body 23, and the like. The metal base material 23c and the metal member 21c are electrically connected to each other, and can both allow an electric current to flow therethrough.

The metal base material 23c and the connector portion 27 are connected to each other via a feedback wiring line 31. The feedback wiring line 31 is arranged running from the inside of the control body 23 of the endoscope 200 through the universal cable 25, and is used to allow an induced electric current generated in the metal member 21c to escape to the frame ground FG. When the endoscope 200 is connected to the processor 100, the feedback wiring line 31 is connected to a relay wiring line 33 of the processor 100 via the connector portion 27. In a state in which the endoscope 200 is connected to the processor 100, the relay wiring line 33 connects the feedback wiring line 31 and a feedback terminal 35 that is provided on an outer cover surface of the processor 100.

A capacitor box 37 in which a capacitor is provided is arranged outside the processor 100. Two wiring lines 37a and 37b extend from the capacitor box 37, and in the capacitor box 37, the two wiring lines 37a and 37b are respectively connected to both ends of the capacitor. Furthermore, the two wiring lines 37a and 37b extending from the capacitor box 37 are respectively detachably connected to the feedback terminal 35 and a ground terminal 39 that are provided on the outer cover surface of the processor 100. The ground terminal 39 is connected to the frame ground FG of the processor 100.

When the endoscope 200 and the capacitor box 37 are connected to the processor 100, the metal member 21c of the insertion portion 21 is connected to the frame ground FG via the metal base material 23c of the control body 23, the feedback wiring line 31, the relay wiring line 33, and the capacitor box 37. The capacitor in the capacitor box 37 usually has the characteristic of letting an AC component of an electric current flow therethrough. Accordingly, as a result of the metal member 21c being grounded via the capacitor box 37 (by AC coupling the metal member 21c to the frame ground FG), it is possible to effectively allow an induced electric current of a high-frequency generated in the metal member 21c to escape to the frame ground FG.

Note that the optimal volume of the capacitor for allowing an induced electric current to escape to the frame ground FG, and the electric circuit configuration of the capacitor box 37 depend on the frequency of the induced electric current. Accordingly, by replacing the capacitor box 37 that is connected to the processor 100 with one that corresponds to an electrosurgical device 400 or a high-frequency power supply 500 that is to be used, it is possible to more reliably allow an induced electric current to escape to the frame ground FG. Furthermore, in the present embodiment, since the capacitor box 37 is detachable from the processor 100 (the feedback terminal 35 and the ground terminal 39), the replacement of the capacitor box 37 is easy.

In the present embodiment, the feedback wiring line 31 is arranged inside the endoscope 200, and is connected to the processor 100 via the connector portion 27. Accordingly, in contrast to the conventional technique, it is not necessary to provide the endoscope 200 of the present embodiment with a feedback terminal that is separate from the connector portion 27. Accordingly, protrusions can be reduced by an amount that corresponds to the feedback terminal that would have protruded from the outer cover of the endoscope 200. Due to the reduction in protrusion from the outer cover of the endoscope 200, it is possible to suppress the insertion portion 21 from coming into contact with the protrusion when, for example, the endoscope 200 is washed or carried. The insertion portion 21 is covered by the protective tube 21b that is made of a soft material. Accordingly, suppressing the insertion portion 21 from coming into contact with the protrusion can suppress damage to the soft protective tube 21b that may be caused by the soft protective tube 21b coming into contact with the protrusion.

Furthermore, in the conventional technique, an endoscope is provided with a connector portion and a feedback terminal that are separate from each other. Accordingly, in order to bring the endoscope into a usable state, it is necessary to perform an operation of connecting the connector portion to a processor, and an operation of connecting a wiring line to the feedback terminal, separately. In contrast, in the present embodiment, the area in which the feedback wiring line 31 is connected to the processor 100 (relay wiring line 33) is arranged in the connector portion 27. Accordingly, by performing an operation of connecting the connector portion 27 to the processor 100, the feedback wiring line 31 is connected to the relay wiring line 33. Accordingly, in the present embodiment, the operation of bringing the endoscope 200 into a usable state is easy, and thus cases where the operator forgets to ground the feedback wiring line 31 can be avoided.

Note that in the configuration shown in FIG. 1, the feedback wiring line 31 is arranged extending from the connector portion 27 of the endoscope 200 to the control body 23, but the present invention is not limited to this configuration. For example, the feedback wiring line 31 may be connected to the metal member 21c of the insertion portion 21. Accordingly, even if the metal base material 23c and the metal member 21c are not connected to each other, it is possible to reliably cause an induced electric current to flow to the frame ground FG.

Furthermore, the metal ember that is used iii the insertion portion 21 is not limited to the coil-shaped metal member 21c. The insertion portion 21 may have, for example, a tubular metal mesh that ranged so as to cover the metal member 21c. This metal mesh is used to block electromagnetic noise that is emitted from the inside of the insertion portion 21 to the outside, and electromagnetic noise that enters the inside of the insertion portion 21 from the outside. In this case, the metal mesh is connected to the metal base material 23c, or both the metal mesh and the metal member 21c are connected to the metal base material 23c.

Furthermore, the capacitor box 37 is connected to the processor 100 if the electrosurgical device 400 is used as a treatment tool. Therefore, if a treatment tool that does not need to be supplied with a high frequency current is used, the capacitor box 37 may be removed from the processor 100.

An exemplary embodiment of the present invention has been described above. The embodiment of the present invention is not limited to the description above, and various modifications are possible within the scope of the technical idea of the present invention. For example, the embodiment of the present invention encompasses content that is obtained by appropriately combining the embodiment and the like that are clearly exemplified in the description, and obvious embodiments and the like.

1. An endoscope system comprising:
a processor, and an endoscope,
the processor including:
a ground terminal that is provided on an outer cover surface of a housing;
a feedback terminal that is provided on the outer cover surface, and is connected to the ground terminal via an external capacitor that is arranged outside the housing; and
a relay wiring line that is connected to the feedback terminal, and is arranged in the housing, and
the endoscope including:
a connector portion that is detachably attached to the processor;
a channel into which a treatment tool is inserted; and
a feedback wiring line that connects a metal member that is located in the vicinity of the channel to the connector portion,
wherein, when the connector portion is attached to the processor, the feedback wiring line and the relay wiring line are connected to each other, and the metal member is connected to the ground terminal via the feedback wiring line, the relay wiring line, the feedback terminal, and the external capacitor.

2. The endoscope system according to claim 1,
wherein the endoscope has a rod-shaped insertion portion that is flexible and is to be inserted into a lumen of a patient, and
the metal member is arranged in the insertion portion, and has a shape of an elongated tube that extends in a longitudinal direction of the insertion portion.

3. The endoscope system according to claim 1,
wherein the external capacitor is detachably connected to the ground terminal and the feedback terminal.
